(19) United States
(12) Patent Application Publication
(10) Pub. No.: US 2004/0241244 A1
(43) Pub. Date: Dec. 2, 2004

(54) Neuronal growth enhancement by prostaglandin compositions and methods

(75) Inventors: Guiting Lin, San Francisco, CA (US); Y. Joseph Mo, Princeton, NJ (US); Mingqi Lu, Lawrenceville, NJ (US); Yinglu Guo, Beijing (CN)

Correspondence Address: BOWDITCH & DEWEY, LLP, 161 WORCESTER ROAD, P.O. BOX 9320, FRAMINGHAM, MA, 01701-9320, US

(73) Assignee: NexMed (Holdings) Inc., Robbinsville, NJ (US)

(21) Appl. No.: 10/803,460

(22) Filed: Mar. 18, 2004

Related U.S. Application Data
(60) Provisional application No. 60/456,597, filed on Mar. 21, 2003.

(51) Int. Cl. A61K 31/557; A61K 9/14

(52) U.S. Cl. 424/486; 514/573

(57) ABSTRACT

The present invention provides compositions and methods of use for treatment of erectile dysfunction associated with neuropathy related to diabetes, prostatectomy, cysto-prostatectomy, radical cystectomy, abdominoperineal resection of the rectum, cryoablation or radiation therapy.

### CROSS REFERENCES TO RELATED APPLICATIONS

The present application claims the benefit of U.S. Provisional Application No. 60/456,597, filed Mar. 21, 2003, the entire contents of which are incorporated herein by reference in their entirety.

### BACKGROUND OF THE INVENTION

Neural control of penile erection involves adrenergic, cholinergic, nonadrenergic and noncholinergic (NANC) neuroeffector systems carried in the cavernous nerves, which are only millimeters away from the capsule of the prostate. Frequently used prostate and bladder cancer treatments, such as cysto-prostatectomy; radical cystectomy, abdominoperineal resection of the rectum, radical prostatectomy, cryoablation, and radiation therapy, are some of the most common causes of erectile dysfunction (ED) in the United States. Such patients exhibit neurogenic erectile dysfunction, thought to arise due to damage to the cavernous nerve that innervates the vascular smooth muscle cells of the corpus cavernosum of the penis.

Radical retropubic prostatectomy (RPP) has been the standard treatment for organ/specimen-confined prostate cancer for several decades, yet erectile dysfunction in selected series is still reported as high as 90% after this procedure, with surgical technique and experience dominant variables influencing outcome (Zippe, C. D., et al., Management of erectile dysfunction following radical prostatectomy, Current Urology Reports, 2: 495-503 (2001). Age is also a factor. Although about 50-70% of younger men regain potency after nerve sparing radical prostatectomy, the potency recovery rate in patients over 70 years of age is less than 10% (Catalona, W. J., et al., Nerve-sparing radical prostatectomy: evaluation of results after 250 patients. J Urol 1990; 143:538-43; discussion 44; Quinlan, D. M., et al., Sexual function following radical prostatectomy: influence of preservation of neurovascular bundles. J Urol 1991; 145:998-1002).

Thus, most men need treatments for erectile dysfunction to be sexually active following radical prostatectomy. Treatments using vacuum constriction devices, intracorporeal injections of vasoactive drugs, and transurethral vasodilators, have reported response rates of 50% to 70%, but poor long-term compliance, having discontinuation rates of nearly 50% at one year.

Nerve-sparing RRP is a technique that seeks to preserve erectile function by avoiding damage to the cavernous nerve bundles adjacent to the prostate. To the extent that erectile function is recovered post-operatively, an interval of several months is usually necessary before patients report the recovery of normal erections (Montorsi F, et al. Recovery of spontaneous erectile function after nerve-sparing radical retropubic prostatectomy with and without early intracavernous injections of alprostadil: results of a prospective, randomized trial. J Urol. 1997;158:1408-1410). Intraoperative electrical stimulation of cavernous nerves resulting in penile tumescence has been used to map the position of nerves during surgery and assess nerve function. However, mapping of cavernous nerve function has not yet been correlated with increased post-operative erectile function.

The corpora cavernosa of the penis are innervated by neurons whose have cell bodies in the pelvic ganglia receive synaptic input from neurons in the sacral parasympathetic nucleus of the spinal cord and contribute their axons as part of the cavernous nerves. The cavernous nerves arise from the pelvic plexus from the lateral surface of the rectum. These nerves run posterolateral to the apex, mid-portion and base of the prostate anterior to Denonvilliers' fascia between the posterolateral surface of the prostate and the rectum to lie between the lateral pelvic fascia and the prostatic fascia. The branches from the cavernous nerve accompany the branches of the prostatovesicular artery and veins in the “neurovascular bundle” that provides a macroscopic landmark for nerve-sparing radical prostatectomy. The cavernous nerve leaves the pelvis between the transverse perineal muscles and membranous urethra before passing beneath the pubic arch to supply each corpus cavernosum. A branch, the lesser cavernous nerve, supplies the corpus spongiosum and penile urethra, and terminates in a delicate network around the erectile tissue. See, generally Rehman, J. & Melman, “Normal Anatomy and Physiology,” pp.1-46 in Mulcahy, J. J., ed., Male Sexual Function: A Guide to Clinical Management, Humana Press, Totowa, N.J., 2001.

The administration of erection effecting and enhancing drugs is taught in U.S. Pat. No. 4,127,118 to LaTorre. This patent teaches a method of treating male impotence by injecting into the penis an appropriate vasodilator, in particular, an adrenergic blocking agent or a smooth muscle relaxant to effect and enhance an erection.

More recently, U.S. Pat. No. 4,801,587 to Voss et al. teaches the application of an ointment to relieve impotence. The ointment consists of the vasodilators papaverine, hydralazine, sodium nitroprusside, phenoxybenzamine, or phentolamine and a carrier to assist absorption of the primary agent through the skin. U.S. Pat. No. 5,256,652 to El-Rashidy teaches the use of an aqueous topical composition of a vasodilator such as papaverine together with hydroxypropyl-β-cyclodextrin.

Prostaglandin E₁ (PGE₁) is a derivative of prostanoic acid, a 20-carbon atom lipid acid, represented by the formula:

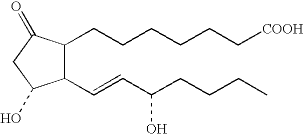

and is commercially available, e.g., from Chinoin Pharmaceutical and Chemical Works Ltd. (Budapest, Hungary) under the designation “Alprostadil USP,” from Pharmacia & Upjohn under the designation “Caverject”. Prostaglandin E₁ complexed with alpha-cyclodextrin is available as alprostatil alfadex from Ono Pharmaceuticals (Japan) and in an injectable form under the designation “Edex®” or “Viradex®” from Schwarz Pharma (Germany). Intracavernosal injection of prostaglandin E₁, alone or in combination with phentolamine and/or papavarine, remains a standard diagnostic and therapeutic for erectile dysfunction. However, scarring and pain at the injection site has reduced patient acceptance of intracavernosal injection as a routine or chronic treatment method.

In one commercially available form (MUSE®, Vivus, Menlo Park Calif.), alprostadil is administered transurethrally as a pellet deposited in the urethra using an applicator with a hollow stem 3.2 cm in length and 3.5 mm in diameter (Padma-Nathan, H., et al., N. Engl. J. Med., 336: 1-7 (1997), see especially FIG. 1). In the home treatment portion of the Padma-Nathan et al. study, 32.7% of the patients (10.8% of administrations) receiving MUSE® complained of penile pain and 5.1% experienced minor urethral trauma, compared to 3.3% and 1.0%, respectively, of the patients receiving placebo. Frequency of report of these side effects has varied in subsequent studies: MUSE® producing penile pain in 17-23.6% of administrations, compared to 1.7% with placebo and minor urethral bleeding reported by 4.8% of patients (Peterson, C. A., et al., J. Urol., 159: 1523-1528 (1998)). In a study on a European population, 31% MUSE® patients reporting penile pain or burning sensations, 4.8% reporting urethral bleeding, and 2.9% reporting severe testicular pain (Porst, H., Int. J. Impot. Res., 9:187-192 (1997)). The percent of patients responding to MUSE® treatment, defined as having at least one erection considered sufficient for intercourse, has been reported to be 43% (Porst, 1997), 65.9% (Padma-Nathan et al., 1997) and 70.5% (Peterson et al., 1998), although published editorial comment has suggested that the percent of patients responding in the latter two studies is more properly reported as 30-40% (Benson, G., J. Urol., 159: 1527-1528 (1998). Intraurethral application of a preparation of 1 mg prostaglandin E₁ in phosphatidylcholine liposomes in 1 ml polyoxyethylene glycol has been reported to be less effective than intracavernosal injection of prostaglandin E₁ (Englehardt, P. F., et al., British J. Urology, 81: 441-444, 1998).

Recently, intrameatal (or meatal) application of a topical PGE₁ composition comprising at least one penetration enhancer has been shown to be a non-invasive alternative to intracavernosal injection or transurethral suppositories for the treatment of erectile dysfunction (see U.S. Pat. No. 6,323,241, the contents of which are hereby incorporated in their entirety). Intrameatal application is the application of medication to the tip of the penis into the navicular fossa by holding the penis upright, holding the meatus open and dropping the medication into the navicular fossa, without introducing the medication container into the meatus.

There have been a few reports of improvement in natural or spontaneous erections in men who use intracavernous injections of papaverine, phenotolamine and/or prostaglandin E₁ for erection induction, but a review of the reports led to a conclusion that the phenomenon occurred at an average prevalence below that of a placebo effect (Sharlip I. D., Does natural erectile function improve following intracavernous injections of vasoactive drugs? Int J Impot Res. 1997 (4): 193-6).

Any recovery of function after nerve-sparing RRP is generally delayed, appearing at about one year after the surgery. One current hypothesis is that ischemic damage due to the hypoxic conditions under reduced oxygenation of the cavernosal tissue is a limiting factor in recovery of erectile function. The hypoxic conditions encourage the development of pathological fibrosis as well as the degeneration of cavernosal smooth muscle. It has been suggested that the oxygenated arterial blood flooding in during an erection would offset the effects of hypoxia. See, generally Novak, T. E., “Management of Erectile Dysfunction Following Radical Prostatectomy,” pp.109-122 in Mulcahy, J. J., ed., Male Sexual Function: A Guide to Clinical Management, Humana Press, Totowa, N.J., 2001.

There have been three brief reports of studies of the effects of intracavernous injections of prostaglandin E₁ after nerve-sparing RRP. In one uncontrolled study, 31 of 40 patients completed the course of treatment (Padma-Nathan, H., et al., The impact on return of spontaneous erections of short-term Alprostadil therapy post nerve sparing prostatectomy, J. Urol. 1997 157 (Suppl. 4): 363 (abstract 1422)). Those who began therapy less than 300 days after surgery had a more positive outcome than those who began therapy more than 300 days after surgery. In a prospective, randomized trial of intracavernous alprostadil injection after nerve sparing RRP, 12 of 15 patients completed the course of treatment (three months of intracavernous alprostadil injections three times a week), and 8 of the 12 reported a recovery of spontaneous erections sufficient for intercourse, compared to 3 of 15 untreated patients (Montorsi F, et al., 1997). The improvement was attributed to improved cavernous oxygenation by the regime of alprostadil injection, limiting the development of hypoxia-induced tissue damage. A third study reported that not only were patients receiving three months of intracavernous alprostadil injections three times a week more likely to recover spontaneous erections, they were also more likely to be responsive to oral sildenafil therapy (Montorsi F, et al., The subsequent use of intracavernous alprostadil and oral sildenafil is more efficacious than sildenafil alone in nerve sparing radical prostatectomy patients, abstract presented at the 2002 annual meeting of the American Urology Association).

The application of prostaglandin E₁ is known to produce extension of neurites in vitro in neuroblastoma cells (Prasad, K. N., Morphological differentiation induced by prostaglandin in mouse neuroblastoma cells in culture, Nature New Biol., 236:49-52 (1972)). Prasad (1972) reported that the optimum concentration of PGE₁ was about 10 micrograms/ml, which corresponds to about 30 micromolar, with an optimum time of exposure of 3-5 days. Prostaglandin E₂ (PGE₂) also produced neurite extension, with the same optimum concentration. See also, Lazo, J. S., Ruddon, R. W., Neurite extension and malignancy of neuroblastoma cells after treatment with prostaglandin E1 and papaverine, J. Natl. Cancer Inst., 59(1):137-43 (1977). “Neurite” is a broad term used to describe both axons and dendrites produced by a nerve cell; most commonly used to cover all cellular processes produced by neurons in culture (Kendrew, J., editor, The Encyclopedia of Molecular Biology, Blackwell Science Ltd., Oxford, 1994, page 709).

The neurite extension effect of PGE₁ in vitro has been shown to be mediated by an increase in intracellular levels of the second messenger cyclic adenosine monophosphate (cAMP). See Prasad, K. N., Neuroblastoma clones: prostaglandin versus dibutyryl cyclic AMP, 8-benzylthio-cyclic AMP, phosphodiesterase inhibitors and x-rays. Proc Soc Exp Biol Med., 140(1):126-9 (1972); Prasad, K. N., & Kumar, S., Role of cyclic AMP in differentiation of human neuroblastoma cells in culture, Cancer, 36(4):1338-43 (1975). The increase in cAMP levels is produced by the binding of PGE₁ or the endogenous ligand PGE₂, to a specific membrane bound receptor of the subclasses EP₂ or EP₄ (Narumiya, S., et al., Prostanoid receptors: Structures, Properties and Functions, Physiological Reviews 79: 1193-1226 (1999)). The affinity of either PGE₁ or PGE₂ for the EP₂ receptor is about 10 nM and for the EP₄ receptor is about 2 nM (Narumiya, S., et al., 1999). Activation of the EP₂ or EP₄ receptors by ligand binding relaxes smooth muscle (Zhang, Y., et al., Characterization of murine vasopressor and vasodepressor prostaglandin E₂ receptors, Hypertension, 35: 1129-1134 (2000)).

The neurite extension effect of PGE₁ or PGE₂ has been shown in other cell lines besides neuroblastoma cells, including a human cells line derived from a primitive neuroectodermal tumor (Fukushige, T., et al., Establishment and characterization of a cell line of congenital primitive neuroectodermal tumor of soft tissue, Virchows Arch. B Cell. Pathol. 62(3):159-66 (1992)), and primary cultures of mouse dorsal root ganglion cells (Hiruma, H., et al., Prostaglandin E₂ enhances axonal transport and neuritogenesis in cultured mouse dorsal root ganglion neurons, Neuroscience, 100(4): 885-91 (2000)). Hiruma et al. reported that 20 micromolar prostaglandin E₂ produced neurite extension in primary dorsal root ganglion cultures at 2 hours that was significantly different from control.

However, none of the above reports disclosed or suggested the possible effects of PGE₁ or PGE₂ on neurite extension of adult postganglionic parasympathetic neurons such as the NANC neurons of the cavernous nerve, either in vivo or in primary cultures in vitro.

In the penile tissue PGE₁ activates cAMP production, thereby inducing smooth muscle relaxation and producing penile erection. PGE₁ has also been postulated to prevent apoptosis via c-Jun N-terminal Kinase (JNK) inactivation and promote neurite outgrowth via cAMP accumulation in cultured neuronal cells (Katoh, H., et al., Prostaglandin E receptor EP3 subtype induces neurite retraction via small GTPase Rho*, J. Biol. Chem. 1996, 271: 29780-29784; Kogawa, S., et al., Apoptosis and impaired axonal regeneration of sensory neurons after nerve crush in diabetic rats. Neuroreport 2000, 11: 663-9). One analogue of PGE₁, OP-1206, has been reported to improve nerve function in diabetic rat after nerve injury by inhibiting apoptosis (Kawamura, T., et al., Prostaglandin E1 transported into cells blocks the apoptotic signals induced by nerve growth factor deprivation. J Neurochem. 1999 72:1907-14; Lando, M., et al., Modulation of intracellular cyclic adenosine monophosphate levels and the differentiation response of human neuroblastoma cells. Cancer Res. 1990 50:722-7; Yasuda, H., et al., Metabolic effect of PGE1 analogue 01206 alpha CD on nerve Na(+)-K(+)-ATPase activity of rats with streptozocin-induced diabetes is mediated via cAMP: possible role of cAMP in diabetic neuropathy. Prostaglandins. 1994 47:367-78). In PC12 cell culture, the retraction of neurites associated with withdrawal of nerve growth factor could be prevented by the addition of PGE₁ (Yasuda, H., et al., A combination of the aldose reductase inhibitor, statil, and the prostaglandin E1 analogue, OP1206. CD, completely improves sciatic motor nerve conduction velocity in streptozocin-induced chronically diabetic rats. Metabolism. 1992 41:778-82; Yamashita, M., et al., Amelioration of nerve Na(+)-K(+)-ATPase activity independently of myo-inositol level by PGE1 analogue OP-1206.alpha-CD in streptozocin-induced diabetic rats. Diabetes. 1991 40:726-30; Lando, M., et al., 1990). Nevertheless, at the present time, there is no direct evidence that PGE₁ can promote cavernous nerve regeneration.

In vivo studies in rats have reported regeneration of cavernous nerve axons at about six months after a lesion (Carrier, S., et al., Regeneration of nitric oxide synthase-containing nerves after cavernous nerve neurotomy in the rat, J Urol. 1995, 153(5): 1722-7; Zhang, X-H., et al., Regeneration of neuronal nitric oxide synthase (nNOS)-containing nerve fibers in rat corpus cavernosum, Asian J. Andrology, 1999, 1: 135-138). Various target-derived growth factors have been suggested as having a possible role in the regeneration of axons in the lesioned cavernous nerve. An in vivo study in rats examined the expression of insulin like growth factor (IGF)-I, nerve growth factor (NGF), transforming growth factor (TGF)-alpha, TGF-beta 1, TGF-beta 2 and TGF-beta 3 and reported a significant increase in expression of IGF-I and TGF-beta 2 (Jung, G. W., et al., The role of growth factor on regeneration of nitric oxide synthase (NOS)—containing nerves after cavernous neurotomy in the rats. Int J Impot Res. 1999, 11(4):227-35; Jung, G. W., et al., IGF-I and TGF-beta2 have a key role on regeneration of nitric oxide synthase (NOS)-containing nerves after cavernous neurotomy in rats. Int J Impot Res. 1999, 11(5):247-59). Gene therapy with brain derived neurotrophic factor has been reported to improve regeneration and intracavernous pressure in vivo in a rat model of neurogenic impotence (Bakircioglu, M. E., et al., The effect of adeno-associated virus mediated brain derived neurotrophic factor in an animal model of neurogenic impotence, J Urol. 2001, 165(6 Pt 1):2103-9).

Another rat study reported an improvement in neurogenic and vasculogenic erectile dysfunction associated with hypercholesterolemia by treatment with vascular endothelial growth factor (VEGF) and adeno-associated virus (AAV) mediated, brain derived neurotrophic factor (BDNF) (Gholami, S. S., et al., The effect of vascular endothelial growth factor and adeno-associated virus mediated brain derived neurotrophic factor on neurogenic and vasculogenic erectile dysfunction induced by hyperlipidemia. J Urol. 2003, 169(4):1577-1581). VEGF has been reported to have neurogenetic and neurotrophic effects in other systems (Sondell, M., et al., Vascular endothelial growth factor has neurotrophic activity and stimulates axonal outgrowth, enhancing cell survival and Schwann cell proliferation in the peripheral nervous system, J Neurosci. 1999, 19(14):5731-40; Sondell, M., et al., Vascular endothelial growth factor is a neurotrophic factor which stimulates axonal outgrowth through the flk-1 receptor, Eur J Neurosci. 2000, 12(12):4243-54; Jin, K., et al., Vascular endothelial growth factor (VEGF) stimulates neurogenesis in vitro and in vivo, Proc Natl Acad Sci USA. 2002, 99(18):11946-50).

Prostaglandins can increase the production of VEGF. PGE₂ has been shown to up-regulate VEGF in vitro in endothelial cells (Pai, R., et al., PGE(2) stimulates VEGF expression in endothelial cells via ERK2/JNK1 signaling pathways. Biochem Biophys Res Commun. 2001, 286(5):923-8.). Treatment of patients with systemic PGE₁ has been reported to up-regulate expression of VEGF (Mehrabi, M. R., et al., Clinical and experimental evidence of prostaglandin E₁-induced angiogenesis in the myocardium of patients with ischemic heart disease, Cardiovasc Res. 2002, 56(2):214-24).

### SUMMARY OF THE INVENTION

In one embodiment, the present invention provides a convenient and non-invasive method of promoting the recovery of spontaneous erectile function after nerve-sparing radical prostatectomy by administering a composition comprising a vasoactive prostaglandin selected from the group consisting of prostaglandin E₁ and prostaglandin E₂ and a penetration enhancer during the first post-operative year to a patient in need of such recovery. In preferred embodiments, the prostaglandin composition is a topical composition comprising prostaglandin E₁ and a penetration enhancer; the topical composition is applied intrameatally to the tip of the penis. Typically, the prostaglandin E₁ is present in an amount generally effective to produce an erection. In other embodiments, the prostaglandin E₁ is present in an amount effective to produce penile tumescence. In preferred embodiments, the prostaglandin composition is administered at least once per week, preferably at least three times per week, in a treatment regime lasting at least one month, preferably lasting at least three months. In a preferred embodiment, the treatment is initiated as soon as tolerated after the operation, preferably before the end of the first postoperative month. The treatment regime is suitably initiated no later than one year after the operation, preferably no later than six months after the operation, most preferably at about one month after the operation.

In other embodiments, the invention provides compositions comprising between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition; a polymer carrier selected from the group consisting of biodegradable polymers and shear-thinning polymeric thickeners; a lipophilic component selected from the group consisting of a C₁ to C₈ aliphatic alcohol, a C₈ to C₃₀ aliphatic ester, a liquid polyol and a mixture thereof; water; and a buffer that provides a buffered pH value for the composition of about 3 to about 7.4. In preferred embodiments, the vasoactive prostaglandin is 0.05 to 1 weight percent prostaglandin E₁, based on the total weight of the composition. In certain preferred embodiments, the biodegradable polymer is flowable at room temperature. The biodegradable polymer is suitably selected from the group consisting of a polylactide, a poly(lactide-co-glycolide), a polyorthoester, a polyphosphazene, a polyanhydrides, and a polyphosphoester. In other embodiments, the biodegradable polymer is a biodegradable triblock copolymer selected from the group consisting of a poly(lactide-co-glycolide)—polyethylene glycol—poly(lactide-co-glycolide) copolymer, a polylactide—polyethylene glycol—polylactide copolymer, a polyethylene glycol—poly(lactide-co-glycolide)—polyethylene glycol copolymer and a polyethylene glycol—polylactide—polyethylene glycol copolymer. Preferably, the shear-thinning polymeric thickener selected from the group consisting of a shear-thinning polysaccharide gum and a shear-thinning polyacrylic acid polymer. When present, the liquid polyol is a polyethylene glycol selected from the group consisting of polyethylene glycol 200, polyethylene glycol 400 and polyethylene glycol 600.

In preferred embodiments, the composition also includes a penetration enhancer selected from the group consisting of an alkyl-(N-substituted amino)alkanoate, an alkyl-2-(N,N-disubstituted amino)alkanoate, an (N-substituted amino)alkanol alkanoate, an (N,N-disubstituted amino)alkanol alkanoate, pharmaceutically acceptable salts thereof and mixtures thereof. The composition can also include an emulsifier, a fragrance or a topical anesthetic.

The present invention also provides methods of promoting the recovery of erectile function in a subject after nerve-sparing radical retropubic prostatectomy comprising the steps of administering during the first post-operative year to the penile meatus of the subject in need of such treatment a topical composition comprising between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition; a shear-thinning polymeric thickener selected from the group consisting of a shear-thinning polysaccharide gum and a shear-thinning polyacrylic acid polymer; a lipophilic component selected from the group consisting of a C₁ to C₈ aliphatic alcohol, a C₈ to C₃₀ aliphatic ester, a liquid polyol and a mixture thereof; water; and a buffer that provides a buffered pH value for the composition of about 3 to about 7.4 and continuing the administration of the topical composition according to a regime of repeated doses.

Typically, the composition further comprises a penetration enhancer selected from the group consisting of an alkyl-(N-substituted amino)alkanoate, an alkyl-2-(N,N-disubstituted amino)alkanoate, an (N-substituted amino)alkanol alkanoate, an (N,N-disubstituted amino)alkanol alkanoate, pharmaceutically acceptable salts thereof and mixtures thereof.

In preferred embodiments, the method further includes the step of placing a drug reservoir in fluid communication with the solution in contact with a cavernous neuron wherein the drug reservoir comprises between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition and a biodegradable polymer. The drug reservoir can be placed at the time of the prostatectomy. In certain embodiments, the biodegradable polymer is selected from the group consisting of a polylactide, a poly(lactide-co-glycolide), a polyorthoester, a polyphosphazene, a polyanhydrides, and a polyphosphoester. In certain embodiments, the biodegradable polymer is a biodegradable triblock copolymer selected from the group consisting of a poly(lactide-co-glycolide)—polyethylene glycol—poly(lactide-co-glycolide) copolymer, a polylactide—polyethylene glycol—polylactide copolymer, a polyethylene glycol—poly(lactide-co-glycolide)—polyethylene glycol copolymer and a polyethylene glycol—polylactide—polyethylene glycol copolymer. Preferably the biodegradable polymer is flowable at room temperature. In preferred embodiments, the solution in contact with the cavernous neuron comprises about 1 micromolar to about 30 micromolar prostaglandin E₁.

In other embodiments, the invention provides a method of enhancing neurite sprouting from a nitric oxide synthase positive pelvic ganglion neuron comprising contacting a portion of the neurons with a solution comprising about 1 micromolar to about 100 micromolar of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition.

Typically, the solution in contact with the nitric oxide synthase positive neuron is in fluid communication with a composition comprising 0.001 weight percent to 1 weight percent of a vasoactive prostaglandin selected from the group consisting of prostaglandin E₁, a pharmaceutically acceptable salt thereof, a lower alkyl ester thereof and mixtures thereof, based on the total weight of the composition and a polymer carrier selected from the group consisting of a biodegradable polymer and a shear-thinning polymeric thickener. The shear-thinning polymeric thickener is selected from the group consisting of a shear-thinning polysaccharide gum and a shear-thinning polyacrylic acid polymer.

The invention also provides a method of promoting the recovery of spontaneous erectile function after nerve-sparing radical retropubic prostatectomy comprising the step of intrameatal administration of a subject in need of such treatment a topical composition comprising between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition; a shear-thinning polymeric thickener selected from the group consisting of a shear-thinning polysaccharide gum and a shear-thinning polyacrylic acid polymer; a lipophilic component selected from the group consisting of a C₁ to C₈ aliphatic alcohol, a C₈ to C₃₀ aliphatic ester, a liquid polyol and a mixture thereof; water; comprising a penetration enhancer selected from the group consisting of an alkyl-(N-substituted amino) alkanoate, an alkyl-2-(N,N-disubstituted amino)alkanoate, an (N-substituted amino) alkanol alkanoate, an (N,N-disubstituted amino)alkanol alkanoate, pharmaceutically acceptable salts thereof and mixtures thereof and a buffer that provides a buffered pH value for the composition of about 3 to about 7.4 and continuing the administration of the topical composition according to a regime of repeated doses during the first year post-operation.

In further embodiments, the invention provides a method for restoring cavernous nerve function in a patient comprising the step of depositing the composition of claim 1 at a site in fluid communication with a cavernous neuron in an amount sufficient to produce an prostaglandin E₁ concentration in the range of about 10 micromolar to about 30 micromolar in the solution contacting the neuronal cell body, axon or axon terminal of the cavernous neuron for a time period of at least about three days. Typically, the cavernous neuron is nitric oxide synthase positive.

In preferred embodiments, the composition comprising the releasable vasoactive prostaglandin and the biocompatible polymer is in fluid communication with the cavernous neuron. The vasoactive prostaglandin can be administered continuously or periodically. The cavernous neuron can be targeted directly by placement of drug reservoir comprising the vasoactive prostaglandin composition adjacent to the pelvic ganglion, pelvic plexus, cavernosal nerve or neurovascular bundle in a compartment that is in fluid communication with the cavernous neuron. In other embodiments, the cavernous neuron can be targeted indirectly, by placing the composition in a compartment that is indirectly (e.g., via vessels of the cardiovascular system or lymphatic system) in fluid communication with the cavernous neuron. Without being held to a particular mechanism, it is believed that the treatment, in one embodiment of the present invention, comprising placing a semisolid prostaglandin composition into the fossa navicularis results in the permeation of prostaglandin E₁ into the tissue of the glans penis and into the corpus spongiosum and the paired corpora cavernosum, and thus into the local circulation.

In other embodiments, the present invention provides a method of promoting the recovery of spontaneous erectile function after cysto-prostatectomy; radical cystectomy, abdominoperineal resection of the rectum, cryoablation, or radiation therapy by administering a composition comprising a prostaglandin during the first post-operative year to a patient in need of such recovery. In another embodiment, the present invention provides a method of promoting the recovery of spontaneous erectile function lost due to diabetic neuropathy comprising administering intrameatally a topical prostaglandin composition comprising a penetration enhancer.

In another embodiment, the present invention provides a method of promoting recovery of spontaneous erectile function after nerve-sparing radical retropubic prostatectomy by implanting a reservoir of releasable prostaglandin adjacent to the pelvic ganglion or pelvic plexus. In another embodiment, a reservoir of releasable prostaglandin is implanted adjacent to the cavernosal nerve or neurovascular bundle. The reservoir of releasable prostaglandin can be implanted in conjunction with other measures, such as nerve grafting, or treatment with growth factors by injection or gene therapy. The reservoir of releasable vasoactive prostaglandin can be prostaglandin in a silicone elastomer capsule, prostaglandin embedded in a flexible matrix or prostaglandin complexed in a cyclodextrin in a suitable implantable permeable container.

In another embodiment, the invention provides a method for restoring cavernous nerve function in a patient which comprises administering to the patient in need of such restoration a prostaglandin E₁ composition in an amount sufficient to produce an extracellular prostaglandin E₁ concentration in the range of about 10 micromolar to about 30 micromolar adjacent to the neuronal cell body, axon or axon terminal of a cavernous neuron of the patient for a time period of at least about three days. The prostaglandin E₁ can be present continuously or periodically. In another embodiment, the invention provides a method for increasing neurite outgrowth from cavernous nerve cells comprising administering a prostaglandin E₁ composition in an amount sufficient to produce an extracellular prostaglandin E₁ concentration in the range of about 10 micromolar to about 30 micromolar adjacent to the neuronal cell body, axon or axon terminal of a cavernous neuron for a time period of at least about three days.

The method of the present invention can be used with other treatments such as nerve grafting or growth factor treatments. In other embodiments, the method of the present invention is a less antigenic alternative to therapies involving peptides or proteins.

Other and further aims, purposes, features, advantages, embodiments and the like will be apparent to those skilled in the art from the present specification and the appended claims.

### BRIEF DESCRIPTION OF THE DRAWINGS

FIG. 1A-F shows photographs of neurite outgrowth in primary cultures of dissected dorsocaudal regions of rat major pelvic ganglia. PGE₁, (Sigma, Inc., USA) was added to the medium at a final concentration of 0 μM (FIG. 1A), 1 μM (FIG. 1B), 10 μM (FIG. 1C), 30 μM (FIG. 1D), 60 μM (FIG. 1E) and 100 μM (FIG. 1F). The ganglia cultures were maintained at 37° C. in a humidified atmosphere with 5% CO₂.

FIG. 2 is a graphical representation of the results of a study of neurite outgrowth produced by culturing dorsocaudal regions of rat major pelvic ganglia in various concentrations of PGE₁.

### DETAILED DESCRIPTION OF THE INVENTION

Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

“Spontaneous erectile function” means the ability to produce an erection, including noctural penile tumescence (NPT) or noctural penile tumescence and rigidity (NPTR), without acute pharmacological intervention.

“Intrameatally” or “meatally” means applying medication to the tip of the penis into the navicular fossa by holding the penis upright, holding the meatus open and dropping the medication into the navicular fossa without introducing the medication container into the meatus.

“Penile tumescence” means the swelling of erectile tissue of the penile, including at least one of the glans, the corpora cavernosa or the corpus spongiosa.

“Cavernous neurons” means neurons that contribute processes, such as axons, to the cavernous nerve.

“Fluid communication” between a drug reservoir and a site of drug action includes transport of the drug through the vessels of the cardiovascular system and the lymphatic system as well as diffusion of the drug through the extracellular fluid.

“Alkyl” means the monovalent linear or branched saturated hydrocarbon radical, consisting solely of carbon and hydrogen atoms, having from one to twenty carbon atoms inclusive, unless otherwise indicated. Examples of an alkyl radical include, but are not limited to, methyl, ethyl, propyl, isopropyl, isobutyl, sec-butyl, tert-butyl, pentyl, n-hexyl, octyl, dodecyl, tetradecyl, eicosyl, and the like.

“Lower alkyl” means the monovalent linear or branched saturated hydrocarbon radical, consisting solely of carbon and hydrogen atoms, having from one to six carbon atoms inclusive, unless otherwise indicated. Examples of a lower alkyl radical include, but are not limited to, methyl, ethyl, propyl, isopropyl, tert-butyl, n-butyl, n-hexyl, and the like.

“Lower alkoxy” means the radical —O—R, wherein R is a lower alkyl radical as defined above. Examples of a lower alkoxy radical include, but are not limited to, methoxy, ethoxy, isopropoxy, and the like.

“Halogen” means the radical fluoro, bromo, chloro, and/or iodo.

“Optional” or “optionally” means that the subsequently described event or circumstance may but need not occur, and that the description includes instances where the event or circumstance occurs and instances in which it does not. For example, “optional bond” means that the bond may or may not be present, and that the description includes single, double, or triple bonds.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” of a compound means a salt that is pharmaceutically acceptable, as defined above, and that possesses the desired pharmacological activity of the parent compound. Such salts include:

1. acid addition salts formed with inorganic acids such as hydrochloric acid, hydrobromic acid, hydrofluoric acid, hydroiodic acid, trifluoroacetic acid, sulfuric acid, nitric acid, phosphoric acid, boric acid and the like; or formed with organic acids such as acetic acid, benzenesulfonic acid, benzoic acid, camphorsulfonic acid, p-chlorobenzenesulfonic acid, cinnamic acid, citric acid, cylcopentanepropionic acid, ethanesulfonic acid, 1,2-ethanedisulfonic acid, formic acid, fumaric acid, glucoheptonic acid, gluconic acid, glutamic acid, glycolic acid, hexanoic acid, heptanoic acid, o-(hydroxybenzoyl)benzoic acid, hydroxynaphtoic acid, 2-hydroxyethanesulfonic acid, lactic acid, lauryl sulfuric acid, maleic acid, malic acid, malonic acid, mandelic acid, methanesulfonic acid, 4-methylbicyclo[2.2.2]oct-2-ene-1-carboxylic acid, 4,4′-methylenebis(3-hydroxy-2-ene-1-carboxylic acid), muconic acid, 2-naphthalenesulfonic acid, oxalic acid, 3-phenylpropionic acid, propionic acid, pyruvic acid, salicylic acid, stearic acid, succinic acid, tartaric acid, tertiary butylacetic acid, p-toluenesulfonic acid, trifluoromethanesulfonic acid, trimethylacetic acid, and the like; or

2. salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, methylamine, ethylamine, hydroxyethylamine, propylamine, dimethylamine, diethylamine, trimethylamine, triethylamine, ethylenediamine, hydroethylamine, morpholine, piperazine, and guanidine and the like. Acceptable inorganic bases include aluminum hydroxide, ammonium hydroxide, calcium hydroxide, potassium hydroxide, sodium carbonate, sodium hydroxide and hydrazine. The preferred pharmaceutically acceptable salts are the salts formed from hydrochloric acid, and trifluoroacetic acid.

“Subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgement of the attending medical or veterinary practitioner, and other factors.

The term “pharmacological effect” as used herein encompasses effects produced in the subject that achieve the intended purpose of a therapy. In one preferred embodiment, a pharmacological effect means that vasospasm symptoms of the subject being treated are prevented, alleviated, or reduced. For example, a pharmacological effect would be one that results in the prevention or reduction of vasospasm in a treated subject.

“Disease state” means any disease, condition, symptom, or indication.

“Treating” or “treatment” of a disease state includes:

1. preventing the disease state, i.e. causing the clinical symptoms of the disease state not to develop in a subject that may be exposed to or predisposed to the disease state, but does not yet experience or display symptoms of the disease state,

2. inhibiting the disease state, i.e., arresting the development of the disease state or its clinical symptoms, or

3. relieving the disease state, ie., causing temporary or progressive regression of the disease state or its clinical symptoms.

“Pro-drug” means a pharmacologically inactive form of a compound which must be metabolized in vivo by a subject after administration into a pharmacologically active form of the compound in order to produce the desired pharmacological effect. After administration to the subject, the pharmacologically inactive form of the compound is converted in vivo under the influence of biological fluids or enzymes into a pharmacologically active form of the compound. Although metabolism occurs for many compounds primarily in the liver, almost all other tissues and organs, especially the lung, are able to carry out varying degrees of metabolism. Pro-drug forms of compounds may be utilized, for example, to improve bioavailability, mask unpleasant characteristics such as bitter taste, alter solubility for intravenous use, or to provide site-specific delivery of the compound. Reference to a compound herein includes pro-drug forms of a compound.

In a preferred embodiment, the pharmaceutical composition comprises at least one vasoactive prostaglandin, preferably prostaglandin E₁, an alkyl (N-substituted amino) ester, a polymer, a lipophilic component, and an acid buffer system.

Vasoactive prostaglandins are those that act as peripheral vasodilators, including naturally occurring prostaglandins such as PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}; semisynthetic or synthetic derivatives of natural prostaglandins, including carboprost tromethamine, dinoprost tromethamine, dinoprostone, lipoprost, gemeprost, metenoprost, sulprostone and tiaprost. Prostaglandin E₁ and prostaglandin E₂ are particularly preferred vasoactive prostaglandins for use in conjunction with the present method.

Additionally, simultaneous administration of one or more non-ecosanoid vasodilators may be desirable and may in some cases exhibit a synergistic effect. The combination of prazosin with prostaglandin E₁ has been found to be particularly advantageous in this regard; the latter drug appears to act as a potentiator for prazosin.

Suitable non-ecosanoid vasodilators include, but are not limited to: nitrates such as nitroglycerin, isosorbide dinitrate, erythrityl tetranitrate, amyl nitrate, sodium nitroprusside, molsidomine, linsidomine chlorhydrate (“SIN-1”) and S-nitroso-N-acetyl-d,l-penicillamine (“SNAP”); amino acids such as L-arginine; long and short acting α-adrenergic blockers such as phenoxybenzamine, dibenamine, phentolamine, tamsulosin and indoramin, especially quinazoline derivatives such as alfuzosin, bunazosin, doxazosin, terazosin, prazosin, and trimazosin; vasodilative natural herbal compositions and bioactive extracts thereof, such as gosyajinki-gan, Satureja obovata, bai-hua qian-hu, lipotab, saiboku-to, vinpocetine, Gingko biloba, bacopa, Gynostemma pentaphyllum, gypenosides, Evodia rutaecarpa, rutaecarpine, dehydroevodiamine, dan-shen, salviae miltiorrhizae radix, shosaikoto, Zizyphi fructus, ginseng and mixtures thereof (U.S. Pat. No. 6,007,824); ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; vasodilators such as nimodepine, pinacidil, cyclandelate, dipyridamole and isoxsuprine; chlorpromazine; haloperidol; yohimbine; trazodone and vasoactive intestinal peptides.

Prostaglandin E₁ is well known to those skilled in the art. Reference may be had to various literature references for its pharmacological activities, side effects, and normal dosage ranges. See for example, Physician's Desk Reference, 51st Ed. (1997), The Merck Index, 12th Ed., Merck & Co., N.J. (1996), and Martindale The Extra Pharmacopoeia, 28th Ed., London, The Pharmaceutical Press (1982). Prostaglandin E₁ as well as other compounds referenced herein are intended to encompass pharmaceutically acceptable derivatives including physiologically compatible salts and ester derivatives thereof.

The quantity of vasoactive prostaglandin, such as prostaglandin E₁, in the pharmaceutical composition is a therapeutically effective amount and necessarily varies according to the desired dose, the dosage form (e.g., suppository or topical), and the particular form of vasoactive prostaglandin used. The term “prostaglandin” as used generically herein refers to the prostaglandin free acid and pharmaceutically acceptable derivatives thereof, including, for example PGE₁, pharmaceutically acceptable salts and lower alkyl esters thereof (the term “lower alkyl” as used herein means straight chain or branched chain alkyl containing one to four carbon atoms). The composition generally contains

When used in combination with a vasoactive prostaglandin, a piperazinyl quinazoline antihypertensive, such as prazosin, is present in the amount of about 0.1 mg to about 2.0 mg per unit dose, depending on the potency of the particular piperazinyl quinazoline antihypertensive and the type and dose of vasoactive prostaglandin used. The dose and the proportion of vasoactive prostaglandin and the piperazinyl quinazoline antihypertensive can be routinely determined by one of ordinary skill without undo experimentation.

Working alone, most drugs, prostaglandin formulations included, do not sufficiently permeate the skin to provide drug concentration levels comparable to those obtained from other drug delivery routes. To overcome this problem, topical drug formulations typically include a skin penetration enhancer. Skin penetration enhancers also may be referred to as absorption enhancers, accelerants, adjuvants, solubilizers, sorption promoters, etc. Whatever the name, such agents serve to improve drug absorption across the skin. Ideal penetration enhancers not only increase drug flux across the skin, but do so without irritating, sensitizing, or damaging skin. Furthermore, ideal penetration enhancers should not adversely affect the physical qualities of the available dosage forms (e.g. cream or gel), or the cosmetic quality of the topical composition.

A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Büyüktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Suitable penetration enhancers for use in prostaglandin topical compositions are disclosed in U.S. Pat. Nos. 4,980,378, 5,082,866 and 6,118,020 and published International Patent Application WO 95/095590, the contents of all of which are incorporated by reference. Topical compositions employing such penetration enhancers for the delivery of prostaglandins are disclosed in U.S. Pat. Nos. 6,046,244, 6,323,241, 6,414,028, and 6,489,207.

The topical composition of the present invention can contain one or more penetration enhancers. Among the preferred penetration enhancers for the present invention are ethanol, propylene glycol, glycerol, ethyl laurate, isopropyl palmitate, isopropyl myristate, laurocapram (Azone™), dioxolanes (described in U.S. Pat. No. 4,861,764), macrocyclic ketones, HP-101, oxazolidones and biodegradable penetration enhancers (described in U.S. Pat. Nos. 4,980,378 and 5,082,866 to Wong et al. such as alkyl-2-(N,N-disubstituted amino)alkanoates (e.g., dodecyl N,N-dimethylamino isoproprionate (DDAIP)), N,N-disubstituted amino alkanol alkanoates) and mixtures thereof. The penetration enhancer is present in an amount sufficient to enhance the penetration of the vasoactive prostaglandin, e.g., prostaglandin E₁. The specific amount varies necessarily according to the desired release rate and the specific form of prostaglandin E₁ used. Generally, the penetration enhancer is present in an amount ranging from about 0.5 weight percent to about 20 weight percent, based on the total weight of the composition. Preferably, the penetration enhancer is present in an amount ranging from about 1 weight percent to about 10 weight percent of the composition. More preferably, the penetration enhancer is present in an amount ranging from about 1 weight percent to about 5 weight percent of the composition.

In general, suitable penetration enhancers can be chosen from those listed above as well as sulfoxides, alcohols, fatty acids, fatty acid esters, polyols, amides, surfactants, terpenes, alkanones, organic acids and mixtures thereof. See generally Chattaraj, S. C. and Walker, R. B., Penetration Enhancer Classification, pp.5-20 in Maibach, H. I., and Smith, H. E., (eds.), Percutaneous Penetration Enhancers, CRC Press, Inc., Boca Raton, Fla. (1995) and Büyüktimkin, N., et al., Chemical Means of Transdermal Drug Permeation Enhancement, in Gosh, T. K., et al., (eds.) Transdermal and Topical Drug Delivery Systems, Interpharm Press, Inc., Buffalo Grove, Ill. (1997). Suitable sulfoxides include dimethylsulfoxide, decylmethylsulfoxide and mixtures thereof. Suitable alcohols include ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, olcyl alcohol, linolyl alcohol, linolenyl alcohol and mixtures thereof. Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol and mixtures thereof.

Suitable amides include urea, dimethylacetamide, diethyltoluamide, dimethylformamide, dimethyloctamide, dimethyldecamide, 1-alkyl-4-imidazolin-2-one, pyrrolidone derivatives, cyclic amides, hexamethylenelauramide and its derivatives, diethanolamine, triethanolamine and mixtures thereof. Suitable pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-decyl-thioethyl-2-pyrrolidone (HP-101), 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkypyrrolidone, fatty acid esters of N-(2-hydroxymethyl)-2-pyrrolidone and mixtures thereof. Suitable cyclic amides include 1-dodecylazacycloheptane-2-one (laurocapram, Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexane-2-one, 1-geranylazacyclopentan-2,5-dione, 1-farnesylazacyclopentan-2-one and mixtures thereof.

Suitable surfactants include anionic surfactants, cationic surfactants, nonionic surfactants, bile salts and lecithin. Suitable anionic surfactants include sodium laurate, sodium lauryl sulfate and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxy-poly(oxyethylene)-poly(oxypropyl)poly(oxyethylene)block copolymers, polyoxyethylene ethers, polyoxyethylene sorbitan esters, polyethylene glycol esters of fatty alcohols and mixtures thereof. Suitable α-hydro-ω-hydroxy-poly(oxyethylene)-poly(oxypropyl) poly(oxyethylene)block copolymers include Poloxamers 231, 182, and 184 and mixtures thereof. Suitable polyoxyethylene ethers include 4-lauryl ether (Brij 30), (Brij 93), (Brij 96), 20-oleyl ether (Brij 99) and mixtures thereof. Suitable polyoxyethylene sorbitan esters include the monolaurate (Tween 20, Span 20) the monopalmitate (Tween 40), the monostearate (Tween 60), and the monooleate (Tween 80) and mixtures thereof. Suitable polyethylene glycol esters of fatty acids include the 8-oxyethylene stearate ester (Myrj 45), (Myrj 51), the 40-oxyethylene stearate ester (Myrj 52) and mixtures thereof. Suitable bile salts include sodium cholate, sodium salts of laurocholic, glycolic and desoxycholic acids and mixtures thereof.

Suitable terpenes include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineole, ylang ylang oil, anise oil, chenopodium oil, eucalyptus oil and mixtures thereof. Suitable alkanones include N-heptane, N-octane, N-nonane, N-decane, N-undecane, N-dodecane, N-tridecane, N-tetradecane, N-hexadecane and mixtures thereof. Suitable organic acids include citric acid, succinic acid, salicylic acid, salicylates (including the methyl, ethyl and propyl glycol derivatives), tartaric acid and mixtures thereof.

In a preferred embodiment, the penetration enhancer is an alkyl-2-(N-substituted amino)-alkanoate, an (N-substituted amino)-alkanol alkanoate, or a mixture of these. For convenient reference, alkyl-2-(N-substituted amino)-alkanoates and (N-substituted amino)-alkanol alkanoates can be grouped together under the label alkyl (N-substituted amino) esters.

Alkyl-2-(N-substituted amino)-alkanoates suitable for the present invention can be represented as follows:

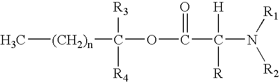

wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

Preferred are alkyl (N,N-disubstituted amino)-alkanoates such as C₄ to C₁₈ alkyl (N,N-disubstituted amino)-acetates and C₄ to C₁₈ alkyl (N,N-disubstituted amino)-propionates and pharmaceutically acceptable salts and derivatives thereof. Exemplary specific alkyl-2-(N,N-disubstituted amino)-alkanoates include dodecyl 2-(N,N dimethylamino)-propionate (DDAIP);

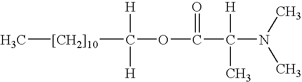

and dodecyl 2-(N,N-dimethylamino)-acetate (DDAA);

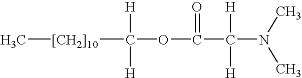

Alkyl-2-(N-substituted amino)-alkanoates are known. For example, dodecyl 2-(N,N-dimethylamino)-propionate (DDAIP) is available from Steroids, Ltd. (Chicago, Ill.). In addition, alkyl-2-(N,N-disubstituted amino)-alkanoates can be synthesized from more readily available compounds as described in U.S. Pat. No. 4,980,378 to Wong et al., which is incorporated herein by reference. As described therein, alkyl-2-(N,N-disubstituted amino)-alkanoates are readily prepared via a two-step synthesis. In the first step, long chain alkyl chloroacetates are prepared by reaction of the corresponding long chain alkanols with chloromethyl chloroformate or the like in the presence of an appropriate base such as triethylamine, typically in a suitable solvent such as chloroform. The reaction can be depicted as follows:

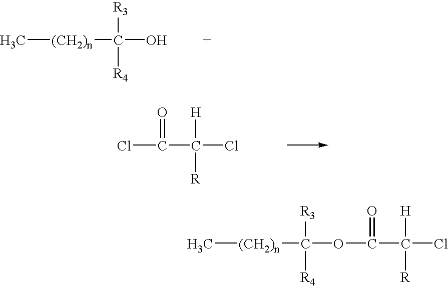

wherein R, R₃, R₄ and n are defined as above. The reaction temperature may be selected from about 10 degrees Celsius to about 200 degrees Celsius or reflux, with room temperature being preferred. The use of a solvent is optional. If a solvent is used, a wide variety of organic solvents may be selected. Choice of a base is likewise not critical. Preferred bases include tertiary amines such as triethylamine, pyridine and the like. Reaction time generally extends from about one hour to three days.

In the second step, the long chain alkyl chloroacetate is condensed with an appropriate amine according to the scheme:

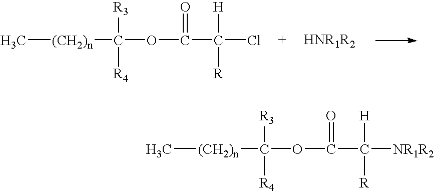

wherein n, R, R₁, R₂, R₃ and R₄ are defined as before. Excess amine reactant is typically used as the base and the reaction is conveniently conducted in a suitable solvent such as ether. This second step is preferably run at room temperature, although temperature may vary. Reaction time usually varies from about one hour to several days. Conventional purification techniques can be applied to ready the resulting ester for use in a pharmaceutical compound.

Suitable (N-substituted amino)-alkanol alkanoates can be represented by the formula:

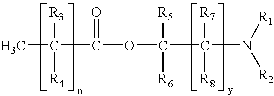

wherein n is an integer having a value in the range of about 5 to about 18; y is an integer having a value in the range of 0 to about 5; and R₁, R₂, R₃, R₄, R₅, R₆, and R₇ are members of the group consisting of hydrogen, C₁ to C₈ alkyl, and C₁ to C₈ aryl; and R₈ is a member of the group consisting of hydrogen, hydroxyl, C₁ to C₈ alkyl, and C₁ to C₈ aryl. The preparation of (N-substituted amino)-alkanol alkanoates and their use as penetration enhancers is disclosed in published PCT International Application WO 95/09590, which is incorporated by reference herein in its entirety.

Preferred are (N-substituted amino)-alkanol alkanoates such as C₅ to C₁₈ carboxylic acid esters and pharmaceutically acceptable salts thereof. Exemplary specific (N,N-disubstituted amino)-alkanol alkanoates include

1-(N,N-dimethylamino)-2-propanol dodecanoate (DAIPD);

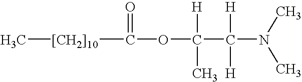

1-(N,N-dimethylamino)-2-propanol myristate (DAIPM);

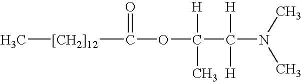

1-(N,N-dimethylamino)-2-propanol oleate (DAIPO);

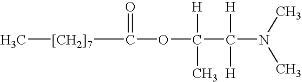

The (N,N-disubstituted amino)-alkanol alkanoates are readily prepared by reacting the corresponding aminoalkinol with lauroyl chloride in the presence of triethylamine. A solvent such as chloroform is optional but preferred. For example, 1-(N,N-dimethylamino)-2-propanol can be reacted with lauroyl chloride in chloroform and in the presence of triethylamine to form 1-(N,N-dimethyl-amino)-2-propanol dodecanoate (DAIPD). Among the suitable penetration enhancers for the present invention DDAIP is generally preferred.

The penetration enhancer is present in an amount sufficient to enhance the penetration of the prostaglandin E₁. The specific amount varies necessarily according to the desired release rate and the specific form of prostaglandin E₁ used. Generally, this amount ranges from about 0.5 percent to about 10 percent, based on the total weight of the composition. In one embodiment, where the vasoactive prostaglandin is prostaglandin E₁, the penetration enhancer is DDAIP in the amount of about 0.01 to about 5 weight percent of the composition.

Additionally, other known transdermal penetration enhancers can also be added, if desired. Illustrative are dimethyl sulfoxide (DMSO), dimethyl acetamide (DMA), 2-pyrrolidone, N,N-diethyl-m-toluamide (DEET), 1-dodecylazacycloheptane-2-one (Azone™, a registered trademark of Nelson Research), N,N-dimethylformamide, N-methyl-2-pyrrolidone, calcium thioglycolate, oxazolidinone, dioxolane derivatives, laurocapram derivatives, and macrocyclic enhancers such as macrocyclic ketones.

Natural and modified polysaccharide gums are also an important ingredient of the composition. Suitable representative gums are those in the natural and modified galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carobtree (ceratonia siliqua). Suitable modified polysaccharide gums include ethers of natural or substituted polysaccharide gums, such as carboxymethyl ethers, ethylene glycol ethers and propylene glycol ethers. An exemplary substituted polysaccharide gum is methylcellulose.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., N.Y. (1980). Most gums are commercially available in various forms, commonly a powder, and ready for use in foods and topical compositions. For example, locust bean gum in powdered form is available from Tic Gums Inc. (Belcam, Md.).

When present, the polysaccharide gums are present in the range from about 0.1 percent to about 5 percent, based on the total weight of the composition, with the preferred range being from 0.5 percent to 3 percent. In one preferred embodiment, 2.5 percent by weight of a polysaccharide gum is present. Illustrative compositions are given in the examples, below.

An optional alternative to the polysaccharide gum is a polyacrylic acid polymer. A common variety of polyacrylic acid polymer is known generically as “carbomer.” Carbomer is polyacrylic acid polymers lightly cross-linked with polyalkenyl polyether. It is commercially available from the B. F. Goodrich Company (Akron, Ohio) under the designation “CARBOPOL™” A particularly preferred variety of carbomer is that designated as “CARBOPOL 940.”

Other polyacrylic acid polymers suitable for use are those commercially available under the designations “Pemulen™” (B. F. Goodrich Company) and “POLYCARBOPHIL™” (A. H. Robbins, Richmond, Va.). The Pemulen™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ enhancer is a polyacrylic acid cross-linked with divinyl glycol. Where polyacrylic acid polymers are present, they represent about 0.5 percent to about 5 percent of the composition, based on its total weight.

In certain preferred embodiments, the semi-solid composition has a suitably chosen viscosity such that the composition is naturally retained within the fossa navicularis. The semi-solid composition can exhibit Newtonian or non-Newtonian rheological characteristics. In some preferred embodiments, the semi-solid composition of the present invention exhibits non-Newtonian rheological characteristics, i.e. in which the apparent viscosity is dependent on the shear rate applied to the composition. Preferably the composition has “shear-thinning” rheological properties. As used herein, “shear-thinning” refers to a reduction in apparent viscosity (the ratio of shear stress to the shear rate) with increasing shear rate, whether the reduction in apparent viscosity is time independent (pseudoplastic), time dependent (thixotropic) or associated with a yield stress, defined as a stress that must be exceeded before flow starts, (Bingham plastics and generalized Bingham plastics). See, generally, Harris, J., & Wilkinson, W. L., “Non-newtonian Fluid,” pp.856-858 in Parker, S. P., ed., McGraw-Hill Encyclopedia of Physics, Second Edition, McGraw-Hill, New York, 1993. A suitable viscosity range of the composition is from about 5,000 centipoise (cps) to about 20,000 cps, preferably from about 7,000 cps to about 13,000 cps.

In preferred embodiments, the vasoactive prostaglandin is released over a period of time from a drug reservoir. While it should be recognized that the release over time of a vasoactive prostaglandin from a semi-solid composition administered meatally and retained within the fossa navicularis is an embodiment of release from a drug reservoir, in other embodiments, the vasoactive prostaglandin can be released from compositions comprising other polymeric carriers that have been placed in other locations. In other preferred embodiments, a composition comprising an effective amount of a vasoactive prostaglandin and a biocompatible polymer is placed in fluid communication with the pelvic ganglion, pelvic plexus or the cavernous nerve. In preferred embodiments, the biocompatible polymer is biodegradable, and preferably flowable at room temperature.

In preferred embodiments, a drug reservoir is formed that comprises a vasoactive prostaglandin and a biocompatible polymer. The biocompatible polymer remains substantially homogenous in the presence of the vasoactive prostaglandin and releases the vasoactive prostaglandin. The biocompatible polymeric material can be hydrophilic or hydrophobic, and can be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, polyolefins, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. The biocompatible polymer may be a protein polymer, fibrin, collagen and derivatives thereof, polysaccharides such as celluloses, starches, dextrans, alginates and derivatives of these polysaccharides, an extracellular matrix component, such as hyaluronic acid, or another biologic agent or a suitable mixture of any of these. The use of an ethylene-vinyl acetate copolymer (EVA, ELVAX-40™, DuPont, Wilmington, Del., USA) and a poly-2-hydroxyethyl-methacrylate polymer (HYDRON™) as drug reservoirs for prostaglandins is known in the art. See, e.g., BenEzra, D., 1978; Form, D. M., & Auerbach, R., 1983; Ziche, M., et al., 1982 and Diaz-Flores, L., et al., Intense vascular sprouting from rat femoral vein induced by prostaglandins E₁ and E₂, Anat Rec., 1994, 238(1):68-76. Such polymers, while biocompatible, have the drawback of requiring removal.

Silicone elastomer drug reservoirs, such as used in Norplant™ (Wyeth) are known in the art. Improvements to drug reservoirs involving modifications of the surface properties of the reservoir are disclosed in U.S. Pat. No. 6,274,159. Preferred silicon elastomers are medical grade silicon elastomers such as SILASTIC™ (Dow-Corning, Midland, Mich.). Such drug reservoirs, while biocompatible, also have the drawback of requiring removal.

In certain preferred embodiments, the drug reservoir is formed from an absorbable or biodegradable polymer. Suitable biodegradable polymers include polylactide (PLA) and poly(lactide-co-glycolide) (PLGA), polyorthoesters, polyphosphazenes, polyanhydrides, and polyphosphoesters. In particularly preferred embodiments, the biodegradable polymer is a polylactide polymer or a poly(lactide-co-glycolide) polymer. Typically the aqueous biodegradable polymer solution is about 9-30% by weight biodegradable copolymer, preferably 20-30% by weight.

The biodegradable polymer comprising the drug reservoir can be a block copolymer. In certain preferred embodiments the polymer is an ABA- or BAB-type block copolymer, where the A-blocks are a relatively hydrophobic poly(lactide-co-glycolide)(PLGA) or hydrophobic poly(lactide)(PLA) and the B-block is a relatively hydrophilic polyethylene glycol (PEG), having a hydrophobic content of between about 51 to 83% by weight and an overall block copolymer molecular weight of between about 2000 and 4990, that exhibit water solubility at low temperatures and undergo reversible thermal gelation at mammalian physiological body temperatures. The making and use of such block copolymers are disclosed in U.S. Pat. No. 6,117,949 and U.S. Published Patent Application No. 20040001872. The biodegradable triblock polymer is typically used in an aqueous solution of about 9-30% by weight copolymer, preferably 20-30% by weight.

In further preferred embodiments, the releasable vasoactive prostaglandin drug reservoir composition is flowable at room temperature and is localized at the deposition site either due to shear-thinning properties or thermal gelation at mammalian physiological body temperatures of the biocompatible polymer.

In preferred embodiments, a solution of a vasoactive prostaglandin in a C₁ to C₈ aliphatic alcohol is added to an aqueous solution of a biodegradable triblock copolymer selected from the group consisting of a PLGA-PEG-PLGA copolymer, a PLA-PEG-PLA copolymer, a PEG-PLGA-PEG copolymer and a PEG-PLA-PEG copolymer to produce a final concentration of 0.001 percent to 1 percent by weight of vasoactive prostaglandin based on the total weight of the composition.

Another important component is a lipophilic component. As used herein “lipophilic component” refers to an agent that is both lipophilic and hydrophilic. One of ordinary skill in the pharmaceutical arts will understand that the lipophilic nature, or “lipophilicity” of a given compound is routinely quantified for comparison to other compounds by using the partition coefficient. The partition coefficient is defined by the International Union of Pure and Applied Chemistry (IUPAC) as the ratio of the distribution of a substance between two phases when the heterogeneous system (of two phases) is in equilibrium; the ratio of concentrations (or, strictly speaking, activities) of the same molecular species in the two phases is constant at constant temperature.

The C₁ to C₈ aliphatic alcohols, the C₂ to C₃₀ aliphatic esters, and their mixtures can serve as lipophilic component. Illustrative suitable alcohols are ethanol, n-propanol and isopropanol, while suitable esters are ethyl acetate, butyl acetate, ethyl laurate, methyl propionate, isopropyl myristate and isopropyl palmitate. As used herein, the term “aliphatic alcohol” includes polyols such as glycerol, propylene glycol and polyethylene glycols. In one embodiment, a mixture of alcohol and ester is preferred, and in particular, a mixture of ethanol and ethyl laurate is preferred.

In some embodiments, the lipophilic component includes at least one liquid polyol. In preferred embodiments, the liquid polyol is a polyethylene glycol selected from the group consisting of polyethylene glycol 200, polyethylene glycol 400 and polyethylene glycol 600. When polyethylene glycol is used, polyethylene glycol is present in the amount of about 1 weight percent to about 25 weight percent, based on the total weight of the composition. A preferred polyethylene glycol is polyethylene glycol 400 (PEG 400). When present, polyethylene glycol 400 is about 1 weight percent to about 25 weight percent, preferably about 3 weight percent to about 20 weight percent, based on the total weight of the composition.

In one embodiment, the C₂ to C₃₀ aliphatic esters, and their mixtures comprising the lipophilic component include C₈ to C₃₀ aliphatic esters of glycerol selected from the group consisting monoglycerides, diglycerides, triglycerides, and mixtures thereof. Suitable aliphatic esters include glyceryl esters of saturated fatty acids, unsaturated fatty acids and mixtures thereof. Suitable saturated fatty acids include caproic acid, caprylic acid, capric acid, lauric acid, myristic acid, palmitic acid, stearic acid, arachidic acid, behenic acid and lignoceric acid. Suitable unsaturated fatty acids include oleic acid, linoleic acid and linolenic acid. Suitable glyceryl esters include glyceryl monooleate, triolein, trimyristin and tristearin, perferably trimyristin.

The concentration of lipophilic component required necessarily varies according to other factors such as the desired semi-solid consistency and the desired skin penetration promoting effects. Suitably the concentration of lipophilic component is in the range of 0.5 percent to 40 percent by weight based on the total weight of the composition. The preferred topical composition contains lipophilic component in the range of 7 percent to 40 percent by weight based on the total weight of the composition.

Where a mixture of aliphatic alcohol and aliphatic ester are employed, the suitable amount of alcohol is in the range of 0.5 percent to 10 percent. In one preferred embodiment, the amount of alcohol is in the range of 5 percent to 15 percent, while that of aliphatic ester is in the range from 2 percent to 15 percent (again based on the total weight of the composition). In another preferred embodiment, the amount of alcohol is in the range of 0.5 percent to 10 percent, while that of aliphatic ester is in the range from 0 percent to 10 percent (again based on the total weight of the composition).

The concentration of lipophilic component required necessarily varies according to other factors such as the desired semi-solid consistency and the desired skin penetration promoting effects. The preferred topical composition contains lipophilic component in the range of 7 percent to 40 percent by weight based on the total weight of the composition. Where a lipophilic component that is a mixture of aliphatic alcohol and aliphatic ester is used, the preferred amount of alcohol is in the range of 5 percent to 15 percent, while that of aliphatic ester is in the range from 2 percent to 15 percent (again based on the total weight of the composition).

An optional, but preferred, component is an emulsifier. Although not a critical factor, a suitable emulsifier generally will exhibit a hydrophilic-lipophilic balance number greater than 10. Sucrose esters, and specifically sucrose stearate, can serve as emulsifiers for the composition. Sucrose stearate is a well-known emulsifier available from various commercial sources. When an emulsifier is used, sucrose stearate is present up to about 2 percent, based on the total weight of the composition, is preferred. The preferred amount of sucrose stearate emulsifier can also be expressed as a weight ratio of emulsifier to polysaccharide gum. A ratio of 1 to 6 emulsifier to gum is preferred, and a ratio of 1 to 4 is most preferred to generate the desired semi-solid consistency and separation resistance.

Other emulsifiers are also suitable including polyoxyethylene sorbitan esters, long chain alcohols, preferably cetostearyl alcohol, and fatty acid glycerides. Suitable polyoxyethylene sorbitan esters include the monolaurate (Tween 20, Span 20) the monopalmitate (Tween 40), the monostearate (Tween 60), and the monooleate (Tween 80) and mixtures thereof. Preferred fatty acid glycerides include glyceryl monooleate, triolein, trimyristin and tristearin.

The composition includes an acid buffer system. Acid buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are countless suitable buffers, potassium phosphate monohydrate has proven effective for compositions of the present invention.

The final pH value of the pharmaceutical composition may vary within the physiologically compatible range. Necessarily, the final pH value is not irritating to human skin or the site of drug reservoir placement. Without violating this constraint, the pH may be selected to improve prostaglandin E₁ stability and to adjust consistency when required. In one embodiment, the preferred pH value is about 3.0 to about 7.4, more preferably about 3.0 to about 6.5, most preferably from about 3.5 to about 6.0.

The remaining component of the composition is water, which is necessarily purified. The composition contains water in the range of about 50 to about 90 percent, based on the total weight of the composition. The specific amount of water present is not critical, however, being adjustable to obtain the desired consistency and/or concentration of the other components.

Prostaglandin E₁ stabilizers, coloring agents, rheological agents, and preservatives can be added to the extent that they do not overly limit prostaglandin E₁ skin penetration or prevent the desired semi-solid consistency.

In preferred embodiments, the dosage forms of the semi-solid pharmaceutical composition are creams, gels, ointments, colloidal suspensions and the like, also including but not limited to compositions suitable for use with transdermal patches and like devices.

The ingredients listed above may be combined in any order and manner that produces a stable composition comprising a prostaglandin E₁ evenly dispersed throughout a semi-solid formulation. One available approach to preparing such compositions involves evenly dispersing the polysaccharide gum (or polyacrylic acid polymer) in a premixed water/buffer solution and then thoroughly homogenizing (i.e. mixing) the resulting mixture, which can be labeled “Part A.” When present, the emulsifier is added to the water/buffer solution before dispersing the polysaccharide gum. Any suitable method of adjusting the pH value of Part A to the desired level may be used, for example, by adding concentrated phosphoric acid or sodium hydroxide.

Separately, the prostaglandin E₁ is dissolved with agitation in the lipophilic component, which itself may be a mixture of alcohols, esters, or alcohol with ester. Next, the penetration enhancer is added. Alternatively, when the lipophilic component includes both an alcohol and an ester, the prostaglandin E₁ can be dissolved in the alcohol before adding the penetration enhancer followed by the ester. In either case, the resulting mixture can be labeled “Part B.” The final step involves slow addition (e.g. dropwise) of Part B into Part A under constant mixing.

The resulting topical composition, when compared to exhibits the advantageous properties described above, including improved prostaglandin E₁ permeation and bioavailability without drug overloading, reduced skin damage and related inflammation, and increased flexibility in design of dosage forms. These compositions can be used for prolonged treatment of peripheral vascular disease, male impotency and other disorders treated by prostaglandin E₁, while avoiding the low bioavailability and rapid chemical decomposition associated with other delivery methods. Application of prostaglandin E₁ in a topical composition to the skin of a patient allows a predetermined amount of prostaglandin E₁ to be administered continuously to the patient and avoids undesirable effects present with a single or multiple administrations of larger dosages by injection. By maintaining a sustained dosage rate, the prostaglandin E₁ level in the patient's target tissue can be better maintained within the optimal therapeutic range.

In one embodiment, a composition comprises about 0.01 percent to about 5 percent modified polysaccharide gum; about 0.001 percent to about 1 percent of a vasoactive prostaglandin selected from the group consisting of PGE₁, pharmaceutically acceptable salts thereof, lower alkyl esters thereof and mixtures thereof; about 0.05 percent to about 10 percent DDAIP or salts thereof; about 0.5 percent to about 10 percent of a lower alcohol selected from the group consisting of ethanol, propanol, isopropanol and mixtures thereof; about 0.5 percent to about 10 percent on an ester selected from the group consisting of ethyl laurate, isopropyl myristate, isopropyl laurate and mixtures thereof; based on the weight of the composition, and an acid buffer. Preferably the composition also comprises up to about 2 percent sucrose stearate.

In preferred drug reservoir embodiments, the vasoactive prostaglandin is 0.05 percent to 1 percent, preferably from 0.1 percent to 0.5 percent prostaglandin E₁, based on the total weight of the composition. Preferably, the biocompatible polymer is selected from the group consisting of a medical grade silicone elastomer, a biodegradable polymer and a shear-thinning polymeric thickener. In preferred embodiments, a solution of prostaglandin E₁ in a C₁ to C₈ aliphatic alcohol is added to an aqueous solution of a biodegradable copolymer. Typically the aqueous biodegradable polymer solution is about 9-30% by weight, preferably 20-30% by weight. If necessary, the pH is adjusted to the preferred pH range of about 3.0 to about 7.4, more preferably about 3.0 to about 6.5, most preferably from about 3.5 to about 6.0. If the biodegradable polymer itself does not provide sufficient buffering capacity to maintain the composition in the desired pH range, a suitable buffer, such as a phosphate buffer, may be added as needed. Typically, the composition also includes a lipophilic component selected from the group consisting of a C₁ to C₈ aliphatic alcohol, a C₈ to C₃₀ aliphatic ester, and a mixture thereof. In preferred embodiments, the composition includes a penetration enhancer selected from the group consisting of an alkyl-2-(N-substituted amino)-alkanoate ester, an (N-substituted amino)-alkanol-alkanoate, or a mixture thereof.

Optionally the composition also comprises up to about 5 percent emulsifier. Preferably, the composition also comprises up to about 2 percent emulsifier. Suitable emulsifiers include polysorbates such as Tweens, glyceryl monooleate, triolein, trimyristin and tristearin. A preferred emulsifier is trimyristin.

The practice of the present invention is demonstrated in the following examples. These examples are meant to illustrate the invention rather than to limit its scope. Variations in the treating compositions which do not adversely affect the effectiveness of prostaglandin E₁ will be evident to one skilled in the art, and are within the scope of this invention. For example, additional ingredients such as coloring agents, anti-microbial preservatives, emulsifiers, perfumes, prostaglandin E₁ stabilizers, and the like may be included in the compositions as long as the resulting composition retains desirable properties, as described above. When present, preservatives are usually added in amounts of about 0.05 to about 0.30%. Suitable preservatives include methylparabens (methyl PABA), propylparabens (propyl PABA) and butylhydroxy toluene (BHT). Suitable perfumes and fragrances are known in the art; a suitable fragrance is up to about 5 percent myrtenol, preferably about 2 percent myrtenol, based on the total weight of the composition.

The topical composition can further include at least one local anesthetic. Suitable local anesthetics include those approved for topical application (“topical anesthetics”), including, but not limited to ambucaine, amolanone, amylocalne hydrochloride, benoxinate, benzocaine, betoxycaine, biphenamine, bupivacaine, butacaine, butamben, butanilicaine, butethamine, butoxycaine, carticaine, chloroprocaine hydrochloride, cocaethylene, cocaine, cyclomethycaine, dibucaine hydrochloride, dimethocaine, diperodon hydrochloride, dyclonine, ecgonidine, ecgonine, ethyl chloride, etidocaine, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocalne, procaine, propanocaine, proparacaine, propipocaine, propoxycaine hydrochloride, pseudococaine, pyrrocaine, ropivacaine, salicyl alcohol, tetracaine hydrochloride, tolycaine, trimecaine, zolamine and mixtures thereof.

When a topical anesthetic is included, the topical anesthetic comprises about 0.01 to about 10% by weight. Typical topical anesthetics include lidocaine, dyclonine, dibucaine, pharmaceutically acceptable salts and mixtures thereof. In one preferred embodiment, the topical anesthetic is about 0.5 to about 1 percent dyclonine, based on the weight of the composition.

The pharmaceutical preparation is preferably in unit dosage form. In such form the preparation is subdivided into unit doses containing appropriate quantities of the active component. The unit dosage form is a packaged preparation, where the package containing the discrete quantities of the pharmaceutical preparation is, e.g. a rigid plastic dispenser or flexible packet.

Another aspect of the invention is an article of manufacture that comprises a composition for treating erectile dysfunction as described above in a suitable container, preferably in a container such as the dispenser disclosed in U.S. Pat. No. 6,224,573, in combination with labeling instructions. Alternatively, the container can be a tube with a suitable orifice size, such as an extended tip tube, pouch, packet, or squeeze bottle and made of any suitable material, for example rigid plastic or flexible plastic.

The labeling instructions can come in the form of a pamphlet, a label applied to or associated with the packaging of the article of manufacture.

The labeling instructions provide for administering a composition of the invention to the meatus of the penis of a patient suffering from erectile dysfunction, directing the patient to hold the penis upright, hold the meatus open and place the composition in the navicular fossa without introducing the container into the meatus about 5-30 minutes, before sexual intercourse. Printed labeling instructions are functionally related to the composition of the invention inasmuch as such labeling instructions describe a method to treat erectile dysfunction according to the present invention. The labeling instructions are an important aspect of the invention in that before a composition can be approved for any particular use, it must be approved for marketing by the responsible national regulatory agency, such as the United States Food and Drug Administration. Part of that process includes providing a label that will accompany the pharmaceutical composition which is ultimately sold. While the label will include a definition of the composition and such other items such as the clinical pharmacology, mechanism of action, drug resistance, pharmacokinetics, absorption, bioavailability, contraindications and the like, it will also provide the necessary dosage, administration and usage. Thus, the combination of the composition with the dispenser with appropriate treatment instructions is important for the proper usage of the drug once it is marketed to the patient. Such treatment instructions will describe the usage in accordance with the method of treatment set forth herein before.

The fossa navicularis is a natural expanded chamber suitably adapted to receive and retain semisolid medicaments. A semi-solid medicament, such as the composition of the present invention, when placed into the meatus has higher impedance to flow at narrowed exits of this space, the meatus and the urethra. The impedance to flow is proportional to the product of the cross sectional area of the path and the path length. Thus, a semi-solid medication of suitably chosen viscosity is naturally retained within the fossa, facilitating the absorption of active agents such as vasodilators and the like. The viscosity of the composition suitably ranges from about 5,000 cps to about 20,000 cps, preferably from about 7,000 cps to about 13,000 cps. In preferred embodiments, the viscosity of the composition is selected so that about 90% to about 99% of the applied composition is retained in the fossa navicularis for up to about thirty minutes. More preferably about 93% to about 98% of the applied composition, optimally more than 98% is retained in the fossa navicularis for up to about thirty minutes.

The quantity of active component in a unit dose preparation may be varied or adjusted from 0.01 mg to 1 g according to the particular application and the potency of the vasoactive prostaglandin. For example, where the vasoactive prostaglandin is prostaglandin E₁, about 0.05 mg to about 0.8 mg prostaglandin E₁ is present, preferably about 0.1 mg to about 0.5 mg and in another embodiment, about 0.2 mg to about 0.3 mg. The composition can, if desired, also contain other compatible therapeutic agents, such as a piperazinyl quinazoline antihypertensive.

Unless otherwise indicated, each composition is prepared by conventionally admixing the respective indicated components together.

### EXAMPLE 1

Exemplary Compositions

Exemplary Composition A was prepared as follows. Part A was formed by dissolving 0.4 parts prostaglandin E₁ (Alprostadil USP) in 5 parts ethyl alcohol. Next, 5 parts dodecyl 2-(N,N-dimethylamino)-propionate were mixed into the alcohol-prostaglandin E₁ solution, followed by 5 parts ethyl laurate.

Part B was prepared starting from a pH 5.5 water/buffer solution. The water/buffer solution was prepared by adding sufficient potassium phosphate monohydride to purified water to create a 0.1 M solution. The pH of the water/buffer solution was adjusted to 5.5 with a strong base solution (1 N sodium hydroxide) and a strong acid (1 N phosphoric acid). The buffer solution represented about 80 parts of the total composition. All parts specified herein are parts by weight.

To the buffer solution, was added 0.5 parts ethyl laurate. Next, the locust bean gum (in powder form) was dispersed in the buffer solution and homogenized using a homogenizer. Table 1, below, contains a list of ingredients.

The resulting composition was a spreadable, semi-solid suitable for application to the skin without the need for supporting devices such as patches and adhesive strips. The composition was both homogenous in appearance and resistant to separation.

TABLE 1
Topical Prostaglandin E₁ Compositions

| Ingredient | | | | | | | | |
|---|---|---|---|---|---|---|---|---|
| (wt %) | A | B | C | D | E | F | G | H |
| prehydrated lo- | 3 | 3 | 3 | 3 | 3 | 3 | 3 | — |
| cust bean gum | | | | | | | | |
| prehydrated | — | — | — | — | — | — | — | 3 |
| modified guar | | | | | | | | |
| gum | | | | | | | | |
| Xanthan gum | — | — | — | — | — | — | — | — |
| water/buffer | 81 | 81 | 81 | 81 | 81 | 81 | 81 | 81 |
| (pH 5.5) | | | | | | | | |
| sucrose stearate | 0.5 | 0.5 | 0.5 | 0.5 | 0.5 | 0.5 | 0.5 | — |
| prostaglandin | 0.1 | 0.2 | 0.3 | 0.4 | 0.4 | 0.5 | 0.4 | 0.3 |
| E₁ | | | | | | | | |
| DDAIP | 5 | 5 | 5 | 5 | 5 | 5 | 5 | 2.5 |
| ethanol | 5 | 5 | 5 | 5 | 5 | 5 | 10 | 5 |
| ethyl laurate | 5 | 5 | 5 | 5 | 5 | 5 | — | 3 |

Additional exemplary compositions B—I are prepared in the same manner using the components listed in Table 1. As noted above, in other embodiments, such as Composition H, the composition may include a modified polysaccharide gum, suitably a modified galactomannan gum, such as a guar gum. Alternatively, a polyacrylic acid polymer may be used instead of the polysaccharide gum.

### EXAMPLE 2

PGE₁ was found to enhance neurite outgrowth in primary cultures of NADPH positive cells derived from dorsocaudal regions of rat major pelvic ganglia. The dorsocaudal regions of 24 major pelvic ganglia (DCR-MPG) were dissected from Sprague-Dawley rats. Each sample was placed on a reduced-growth factor Matrigel coated glasscover slide in a culture dish filled with serum-free medium. PGE₁ (Sigma) was added to the medium at a final concentration of 1, 10, 20, 30, 60, or 100 micromoles (μM). The ganglia cultures were incubated at 37° C. in a humidified atmosphere with 5% CO₂. Digital photographs were taken of neurite growth after 96 hours. The control group (6 samples) was incubated in Matrigel in serum free medium without PGE₁.

Growth Factor Reduced Matrigel (Passaniti, A., et al., Lab. Invest. 1992 67:518-528) was purchased from Becton Dickinson (Mountain View, Calif.). Cell culture grade PGE₁ was purchased from Sigma Chemical. (St. Louis, Mo.). RPIM-1640 and other cell culture reagents were purchased from GIBCO Invitrogen Corp. (Grand Island, N.Y.).

Male Sprague-Dawley rats, two-months old, were used in this study. All animal care, treatments, and procedures were approved by the institutional Committee on Animal Research. The rats were sacrificed by intraperitoneal injection of sodium pentobarbital (50 mg/kg) followed by bilateral thoracotomy and the removal of the dorsocaudal region of the major pelvic ganglia (DCR-MPG).

Each rat supplied a pair of DCR-MPG; one was used as control and the contralateral ganglion was treated with PGE₁. The DCR-MPG isolated from Sprague-Dawley rats were cultured attached to Matrigel-coated glass coverslips. The coverslips were used as the supporting platform to facilitate samples processing for histological staining and examination. Coverslips were coated as follows. Growth Factor Reduced Matrigel (Becton Dickinson, Mountain View, Calif.) was diluted 3-fold in serum-free RPMI-1640 in a 35-mm culture dish on ice. The diluted Matrigel was then spread onto cold sterilized glass coverslips using a sterilized glass slide as spreader. The coated coverslips were placed in 35-mm culture dishes and incubated at 37° C. for 1 hr to allow the Matrigel to solidify. Each freshly dissected DCR-MPG was rinsed in phosphate-buffered saline (PBS), cut into equal thirds, placed on top of Matrigel coated coverslips and covered by 50 μl cold Growth Factor Reduced Matrigel which had been kept in liquid form. After a 5-min incubation at 37° C. to allow the Matrigel to polymerize, 3 ml of serum-free RPMI 1640 medium supplemented with 1×penicillin-streptomycin-fungizone (Cell Culture Facility, University of California, San Francisco) was added. PGE₁ (Sigma, Inc., USA) was added to the medium at a final concentration of 1, 10, 20, 30, 60 and 100 μM. The ganglia cultures were maintained at 37° C. in a humidified atmosphere with 5% CO₂.

After 2 to 3 days of incubation, the ganglia and outgrowing neurites were stained for nicotinamide adenine dinucleotide phosphate (NADPH) diaphorase as an indication of nitric oxide synthase (NOS) expression. The culture medium was removed from each culture and replaced with a fixative solution of 2% formaldehyde, 0.002% picric acid in 0.1 M phosphate buffer, pH 8.0. After 20 min of fixation, the tissue was rinsed three times, 10 min each, with phosphate buffer, pH 8.0. The tissue was then incubated with 0.1 mM NADPH, 0.2 mM nitroblue tetrazolium, 0.2% Triton X-100 in buffer for approximately 30 min at room temperature with constant microscopic monitoring for color development. When a deep blue color was detected for NADPH-diaphorase positive nerves, the reaction was terminated by washing the tissues with buffer three times, 10 min each. The coverslips containing the ganglia were then mounted onto glass slides using buffered glycerin as mounting medium.

Neurites were photographed using a professional DCS-420 digital camera (Eastman Kodak, Rochester, N.Y.) connected to an Olympus microscope and an Apple Macintosh PowerMac computer. All samples were photographed and the images were stored for later analysis. The digital images were analyzed using Chemilmager 4000 software (Alpha Innotech Corporation, San Leandro, Calif.) to determine the maximum length of the outgrowing neurites fibers.

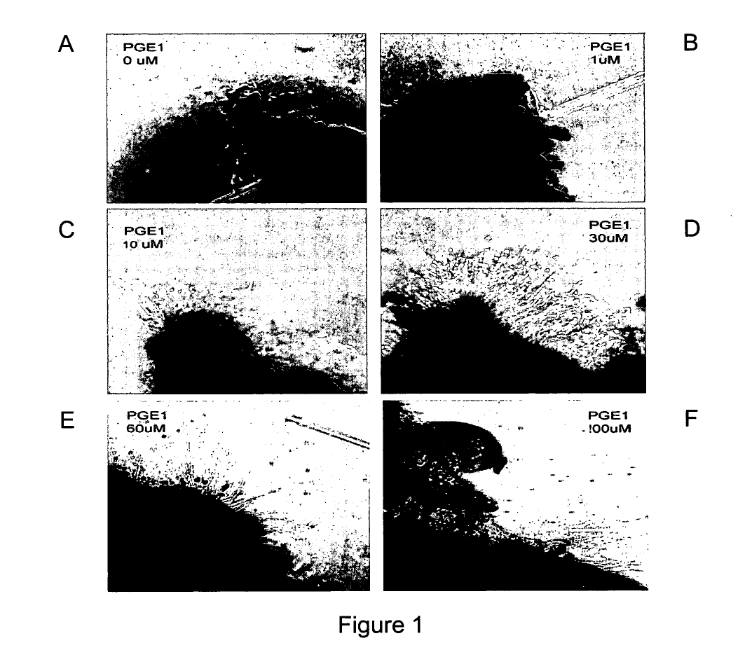

Statistical analysis was performed using computer software from Primer of Biostatistics, 3^{rd} ed. (Glantz SA, McGraw-Hill, Inc. New York, 1992). The data involving different time points were first analyzed by one-way analysis of variance (ANOVA). If ANOVA indicated a significant difference, the Student-Neuman-Keuls test was used to perform pair-wise comparisons. The results are shown in FIG. 1.

### EXAMPLE 3

Four pairs of DCR-MPG were isolated and cultured as described in Example 2. The left side DCR-MPG were treated with PGE₁ at a final concentration of 10 μM and the right served as controls. After 96 hr of culturing, significant outgrowth of neurites could be seen in PGE₁ treatment DCR-MPGs when compared with the controls. The maximum neurite length of PGE₁ treatment was 229.33±10.7 μm and the control was 93.33±28.4 μm (P<0.001).

Eight pairs of DCR-MPG were used in a study to explore the relationship between PGE₁ dose and neurite outgrowth. PGE₁ was added to the medium at a final concentration of 1, 10, 20, 30, 60 or 100 μM. See Table 2, below. The maximum PGE₁ effect was observed at 30 μM PGE₁, which induced 250±38.1 μM of fiber length. The difference is significant (P<0.05).

TABLE 2
Neurite Growth
(Length of longest neurite, μm)

| PGE₁ | | Standard | | |
|---|---|---|---|---|
| Concentration | Mean (μm) | Deviation | S.E.M. | N |
| 0 μM | 93.33 | 39.46 | 22.78 | 3 |
| 1 μM | 168.00 | 16.00 | 6.53 | 6 |
| 10 μM | 229.33 | 18.48 | 10.66 | 3 |
| 20 μM | 244.00 | 26.17 | 10.68 | 6 |
| 30 μM | 250.29 | 62.77 | 23.43 | 7 |
| 60 μM | 138.00 | 28.75 | 14.38 | 4 |
| 100 μM | 112.00 | 42.33 | 24.44 | 3 |

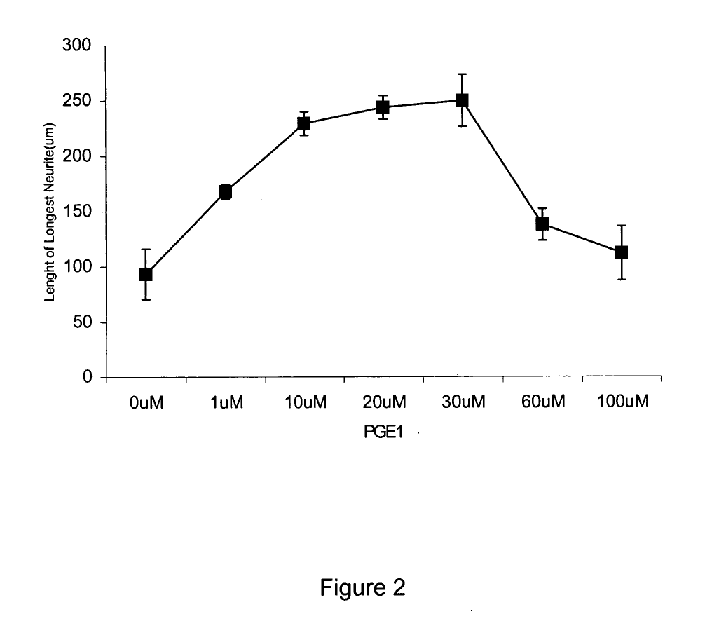

The results are summarized in FIG. 2. There is a dose-dependent effect, with peak neurite growth of 250±63 (mean standard±deviation) micrometers at 30 μM PGE₁. High doses (60 μM, 100 μM) produced a smaller maximum neurite extension.

### EXAMPLE 4

Post-Operative Treatment of Patients after Nerve-Sparing RPP.

A semi-solid prostaglandin topical composition, such as Composition H, is used to promote the recovery of spontaneous erectile function after nerve-sparing radical retropubic prostatectomy in a group of patients needing such treatment. Treatment is started during the first post-operative year, typically beginning, if appropriate, as early as the one-month follow-up visit after surgery. Treatment is performed according to a regime of periodic intrameatal administration of the prostaglandin topical composition at least once weekly, preferably at least three times weekly, for a period of at least three months, preferably at least six months. Each patient is instructed to place the medication in the navicular fossa by holding the penis upright, holding the meatus open and dropping the medication into the navicular fossa without introducing the medication container into the meatus. Recovery of spontaneous erectile function is observed after 3-6 months treatment.

### EXAMPLE 5

Combination Post-Operative Treatment of Patients after Nerve-Sparing RPP.

Combination therapy involving an implantable prostaglandin reservoir in conjunction with meatal administration of a semi-solid prostaglandin topical composition, such as Composition H, is used to promote the recovery of spontaneous erectile function after nerve-sparing radical retropubic prostatectomy in a group of patients needing such treatment.

A drug reservoir comprising a vasoactive prostaglandin and a biocompatible polymer is placed in fluid communication with the pelvic plexus and/or the cavernous nerve during the prostatectomy. In preferred embodiments, the biocompatible polymer is biodegradable. In preferred embodiments, the biocompatible polymer is flowable at room temperature.

Meatal treatment is started during the first post-operative year, typically beginning, if appropriate, as early as the one-month follow-up visit after surgery. Treatment is performed according to a regime of periodic meatal administration of the prostaglandin topical composition at least once weekly, preferably at least three times weekly, for a period of at least three months, preferably at least six months. Each patient is instructed to place the medication in the navicular fossa by holding the penis upright, holding the meatus open and dropping the medication into the navicularfossa without introducing the medication container into the meatus. Recovery of spontaneous erectile function is observed after 3-6 months treatment.

### EXAMPLE 6

Treatment of Patients with Erectile Dysfunction Associated with Diabetic Neuropathy

Intrameatal application of a topical composition comprising a vasoactive prostaglandin and a penetration enhancer provides a method of promoting the recovery of spontaneous erectile function in patients with erectile dysfunction associated with diabetic neuropathy. A topical prostaglandin composition, suitably composition H of Example 1, is provided to the patient in need of treatment. Each patient is instructed to apply the medication intrameatally, i.e. applying medication to the tip of the penis into the navicular fossa by holding the penis upright, holding the meatus open and dropping the medication into the navicular fossa without introducing the medication container into the meatus. The topical composition provides a tumescence-inducing, preferably an erection-inducing, dose of PGE₁. Treatment is performed according to a regime of periodic intrameatal administration of the topical prostaglandin composition at least once weekly, preferably at least thrice weekly, for a period of at least three months, preferably at least six months.

Intrameatal application of the topical prostaglandin composition produces penile tumescence, and in the majority of applications, an erection sufficient for vaginal penetration. Recovery of spontaneous erectile function is observed after 3-6 months treatment.

While the foregoing is intended to be illustrative of the present invention, the scope is defined by the appended claims. Numerous variations and modifications may be effected without departing from the true spirit and scope of the invention.

We claim:

1. A composition comprising
between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition;
a polymer carrier selected from the group consisting of a medical grade silicone elastomer, a biodegradable polymer and a shear-thinning polymeric thickener;
a lipophilic component selected from the group consisting of a C₁ to C₈ aliphatic alcohol, a C₈ to C₃₀ aliphatic ester, a liquid polyol and a mixture thereof;
water; and
a buffer that provides a buffered pH value for the composition of about 3 to about 7.4.

2. The composition of claim 1 wherein the vasoactive prostaglandin is 0.05 to 1 weight percent prostaglandin E₁, based on the total weight of the composition.

3. The composition of claim 1 wherein the polymer carrier is a biodegradable polymer that is flowable at room temperature.

4. The composition of claim 1 wherein the polymer carrier is a biodegradable polymer that is selected from the group consisting of a polylactide, a poly(lactide-co-glycolide), a polyorthoester, a polyphosphazene, a polyanhydrides, and a polyphosphoester.

5. The composition of claim 1 wherein the polymer carrier is a biodegradable polymer that is a biodegradable triblock copolymer selected from the group consisting of a poly(lactide-co-glycolide)—polyethylene glycol—poly(lactide-co-glycolide) copolymer, a polylactide—polyethylene glycol—polylactide copolymer, a polyethylene glycol—poly(lactide-co-glycolide)—polyethylene glycol copolymer and a polyethylene glycol—polylactide—polyethylene glycol copolymer.

6. The composition of claim 1 wherein the polymer carrier is a shear-thinning polymeric thickener that is selected from the group consisting of a shear-thinning polysaccharide gum and a shear-thinning polyacrylic acid polymer.

7. The composition of claim 1 wherein the liquid polyol is a polyethylene glycol selected from the group consisting of polyethylene glycol 200, polyethylene glycol 400 and polyethylene glycol 600.

8. The composition of claim 1 further comprising a penetration enhancer selected from the group consisting of an alkyl-(N-substituted amino)alkanoate, an alkyl-2-(N,N-disubstituted amino)alkanoate, an (N-substituted amino)alkanol alkanoate, an (N,N-disubstituted amino)alkanol alkanoate, pharmaceutically acceptable salts thereof and mixtures thereof.

9. The composition of claim 1 further comprising an emulsifier.

10. The composition of claim 1 further comprising a fragrance.

11. The composition of claim 1 further comprising a topical anesthetic.

12. A method of promoting the recovery of erectile function in a subject after nerve-sparing radical retropubic prostatectomy comprising:
administering during the first post-operative year to the penile meatus of the subject in need of such treatment a topical composition comprising between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition;
a shear-thinning polymeric thickener selected from the group consisting of a shear-thinning polysaccharide gum and a shear-thinning polyacrylic acid polymer;
a lipophilic component selected from the group consisting of a C₁ to C₈ aliphatic alcohol, a C₈ to C₃₀ aliphatic ester,
a liquid polyol and a mixture thereof;
water; and
a buffer that provides a buffered pH value for the composition of about 3 to about 7.4; and
continuing the administration of the topical composition according to a regime of periodic doses.

13. The method of claim 12 wherein the composition further comprises a penetration enhancer selected from the group consisting of an alkyl-(N-substituted amino)alkanoate, an alkyl-2-(N,N-disubstituted amino)alkanoate, an (N-substituted amino)alkanol alkanoate, an (N,N-disubstituted amino)alkanol alkanoate, pharmaceutically acceptable salts thereof and mixtures thereof.

14. The method of claim 12 further comprising the step of placing a drug reservoir in fluid communication with the solution in contact with a cavernous neuron wherein the drug reservoir comprises between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition and a polymer carrier that is selected from the group consisting of a medical grade silicone elastomer and a biodegradable polymer.

15. The method of claim 14 wherein the drug reservoir is placed at the time of the prostatectomy.

16. The method of claim 12 wherein the vasoactive prostaglandin is 0.05 to 1 weight percent prostaglandin E₁, based on the total weight of the composition.

17. The method of claim 14 wherein the polymer carrier is a biodegradable polymer that is selected from the group consisting of a polylactide, a poly(lactide-co-glycolide), a polyorthoester, a polyphosphazene, a polyanhydrides, and a polyphosphoester.

18. The method of claim 14 wherein the polymer carrier is a biodegradable polymer that is a biodegradable triblock copolymer selected from the group consisting of a poly(lactide-co-glycolide)—polyethylene glycol—poly(lactide-co-glycolide) copolymer, a polylactide—polyethylene glycol—polylactide copolymer, a polyethylene glycol—poly(lactide-co-glycolide)—polyethylene glycol copolymer and a polyethylene glycol—polylactide—polyethylene glycol copolymer.

19. The method of claim 14 wherein the polymer carrier is a biodegradable polymer that is flowable at room temperature.

20. The method of claim 14 wherein the solution in contact with the cavernous neuron comprises at least 1 micromolar prostaglandin E₁.

21. The method of claim 14 wherein the solution in contact with the cavernous neuron comprises about 1 micromolar to about 30 micromolar prostaglandin E₁.

22. A method of enhancing neurite sprouting from a pelvic ganglion neuron that is nitric oxide synthase positive comprising contacting at least a portion of the neuron with a solution comprising about 1 micromolar to about 100 micromolar of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition.

23. The method of claim 22 wherein the solution in contact with the nitric oxide synthase positive neuron is in fluid communication with a composition comprising 0.001 weight percent to 1 weight percent of a vasoactive prostaglandin selected from the group consisting of prostaglandin E₁, a pharmaceutically acceptable salt thereof, a lower alkyl ester thereof and mixtures thereof, based on the total weight of the composition and a polymer carrier selected from the group consisting of a medical grade silicone elastomer, a biodegradable polymer and a shear-thinning polymeric thickener.

24. The method of claim 23 wherein the polymer carrier is a shear-thinning polymeric thickener selected from the group consisting of a shear-thinning polysaccharide gum and a shear-thinning polyacrylic acid polymer.

25. A method of promoting the recovery of spontaneous erectile function after nerve-sparing radical retropubic prostatectomy in a subject in need of such treatment comprising the steps of:
administering to the penile meatus a topical composition comprising
between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition;
a shear-thinning polymeric thickener selected from the group consisting of a shear-thinning polysaccharide gum and a shear-thinning polyacrylic acid polymer;
a lipophilic component selected from the group consisting of a C₁ to C₈ aliphatic alcohol, a C₈ to C₃₀ aliphatic ester, a liquid polyol and a mixture thereof; water; and
a penetration enhancer selected from the group consisting of an alkyl-(N-substituted amino)alkanoate, an alkyl-2-(N,N-disubstituted amino) alkanoate, an (N-substituted amino)alkanol alkanoate, an (N,N-disubstituted amino)alkanol alkanoate, pharmaceutically acceptable salts thereof and mixtures thereof and a buffer that provides a buffered pH value for the composition of about 3 to about 7.4; and
continuing the administration of the topical composition according to a regime of periodic doses during the first year post-operation.

26. A method for restoring cavernous nerve function in a patient comprising the step of depositing the composition of claim 1 at a site in fluid communication with a cavernous neuron in an amount sufficient to produce an prostaglandin E₁ concentration in the range of at least 1 micromolar in the solution contacting the neuronal cell body, axon or axon terminal of the cavernous neuron for a time period of at least about three days.

27. A method for restoring cavernous nerve function in a patient comprising the step of depositing the composition of claim 1 at a site in fluid communication with a cavernous neuron in an amount sufficient to produce an prostaglandin E₁ concentration in the range of about 10 micromolar to about 30 micromolar in the solution contacting the neuronal cell body, axon or axon terminal of the cavernous neuron for a time period of at least about three days.

28. The method of claim 26 wherein the cavernous neuron is nitric oxide synthase positive.

29. The method of claim 27 wherein the polymer carrier is a shear-thinning polymeric thickener and the site is the fossa navicularis.

30. The method of claim 29 wherein the composition further comprises a penetration enhancer selected from the group consisting of an alkyl-(N-substituted amino)alkanoate, an alkyl-2-(N,N-disubstituted amino)alkanoate, an (N-substituted amino)alkanol alkanoate, an (N,N-disubstituted amino)alkanol alkanoate, pharmaceutically acceptable salts thereof and mixtures thereof.

31. A method of treatment of erectile dysfunction associated with prostatectomy, cysto-prostatectomy, radical cystectomy, abdominoperineal resection of the rectum, cryoabalation or radiation therapy comprising the step of placing the composition of claim 1 in a drug reservoir in fluid communication with the solution in contact with a portion of a cavernous neuron.

32. A method of treatment of erectile dysfunction associated with prostatectomy, cysto-prostatectomy, radical cystectomy, abdominoperineal resection of the rectum, cryoabalation or radiation therapy comprising the step of placing the composition of claim 6 in a drug reservoir in fluid communication with the solution in contact with a portion of a cavernous neuron.

33. A method of treatment of erectile dysfunction associated with prostatectomy, cysto-prostatectomy, radical cystectomy, abdominoperineal resection of the rectum, cryoabalation or radiation therapy comprising the step of placing the composition of claim 8 in a drug reservoir in fluid communication with the solution in contact with a portion of a cavernous neuron.

34. A method of treatment of erectile dysfunction associated with neuropathy comprising the step of
administering to the penile meatus of the subject in need of such treatment a topical composition comprising between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition;
a shear-thinning polymeric thickener selected from the group consisting of a shear-thinning polysaccharide gum and a shear-thinning polyacrylic acid polymer;
a lipophilic component selected from the group consisting of a C₁ to C₈ aliphatic alcohol, a C₈ to C₃₀ aliphatic ester,
a liquid polyol and a mixture thereof;
water; and
a buffer that provides a buffered pH value for the composition of about 3 to about 7.4; and
continuing the administration of the topical composition according to a regime of periodic doses.

35. The method of claim 34 wherein the neuropathy is diabetic neuropathy.
